(19) United States
(12) Patent Application Publication
(10) Pub. No.: US 2021/0153507 A1
(43) Pub. Date: May 27, 2021

(54) A Strain Of Bactericidal Nitrogen-Fixing Pseudomonas Protegens, A Fermentation Method And An Application Thereof

(71) Applicants: Shandong University, Jinan City, Shandong (CN); Dezhou Microp Bio-Technology Co., Ltd., Yucheng City, Shandong (CN)

(72) Inventors: Youming Zhang, Jinan City, Shandong (CN); Qiang Tu, Jinan City, Shandong (CN); Fangnan Yu, Yucheng City, Shandong (CN); Xiaoshu Jing, Jinan City, Shandong (CN); Xiaoying Bian, Jinan City, Shandong (CN); Hanna Chen, Jinan City, Shandong (CN)

(21) Appl. No.: 16/759,077

(22) PCT Filed: Aug. 30, 2018
(86) PCT No.: PCT/CN2018/103224

§ 371 Date: Apr. 24, 2020

(30) Foreign Application Priority Data
Oct. 27, 2017 (CN) 201711027268.4

(51) Int. Cl. A01N 63/27; C12R 1/38; C12N 1/20

(52) U.S. Cl. CPC A01N 63/27; C12N 1/20; C12R 1/38

(57) ABSTRACT

Provided is a strain of bactericidal nitrogen-fixing Pseudomonas protegens CHA0-ΔretS-NiF, a fermentation method and an application thereof. The strain is deposited under the accession number CGMCC No. 14476. The optimum culture condition thereof is at pH 7, the temperature of 28° C., and rotation speed of 600 rpm. Further provided is a microbial agent containing the Pseudomonas protegens CHA0-ΔretS-NiF as an active ingredient. The Pseudomonas protegens CHA0-ΔretS-NiF has strong nitrogen-fixing and bactericidal capabilities and may be used to prevent and cure plant diseases and to boost plant growth.

### FIELD OF THE INVENTION

The invention belongs to the field of biotechnology. In particular, the present invention relates to a strain of bactericidal nitrogen-fixing Pseudomonas protegens, and the fermentation method and application thereof, and particularly to its application in biological control.

### BACKGROUND

Pseudomonas protegens is a plant biocontrol bacterium that can secrete a variety of active substances, and has certain effects in anti-bacterial, fungal, soil-dwelling insect larvae aspects, etc. Therefore, it has great development prospects in plant disease control and has the potential to replace chemical pesticides.

Pseudomonas protegens CHA0 is isolated from tobacco roots, and produces the secondary metabolite 2,4-diacetylphloroglu-cinol (2,4-DAPG) which can effectively control wheat total erosion caused by Gaeu-mannomyces graminis var. tritici, tobacco black root rot caused by 20 Thielaviopsis basicola, and tomato bacterial wilt caused by Ralstonia solanacearum. retS gene is a negative regulator of the secondary metabolite 2,4-DAPG secreted by and a related red pigment synthesis.

In recent years, due to the excessive use of chemical fertilizers, the soil mechanism has changed, and the current status of continuous cropping obstacles, secondary salinization, compaction and acidification has greatly hindered the increase of crop yields. Among them, nitrogen in fertilizers is an indispensable nutrient element for plants, and the most important way of nitrogen input in nature is biological nitrogen fixation. Studies have shown that nitrogen-fixing microorganisms can effectively provide plants with nitrogen nutrients for their absorption and utilization, and promote their growth.

### SUMMARY OF THE INVENTION

The purpose of the present invention is to overcome the shortcomings of the prior art and provide a mutant strain of Pseudomonas protegens having bactericidal and nitrogen-fixing capabilities. Using biological engineering means, retS gene in the wild-type Pseudomonas protegens CHA0 is knocked out, and the nitrogen-fixing gene cluster NiF is incorporated, and finally the bactericidal nitrogen-fixing engineering bacterium CHA0-ΔretS-NiF is obtained.

Specifically, the present invention provides Pseudomonas protegens CHA0 mutant strain CHA0-ΔretS-NiF, which is deposited under the accession number CGMCC No. 14476.

The present invention also provides a composition, characterized in that its active ingredient is the Pseudomonas protegens mutant strain CHA0-ΔretS-NiF. The composition may be a microbial agent.

The invention also relates to use of the Pseudomonas protegens mutant strain CHA0-ΔretS-NiF in killing bacteria in plants, fixing nitrogen, promoting plant growth, increasing plant yield, and/or controlling plant diseases.

In another aspect, the present invention provides a method for producing the Pseudomonas protegens mutant strain CHA0-ΔretS-NiF, which includes the following steps:

a) Knocking out retS gene in the genome of Pseudomonas protegens CHA0; and

b) Cloning the entire NiF nitrogen-fixing gene island in the genome of Pseudomonas stutzeri DSM4166 into the strain obtained in step a), and then heterologously expressing the same.

The invention further relates to a method for killing bacteria in plants, fixing nitrogen, promoting plant growth, increasing plant yield, and/or controlling plant diseases, comprising administering to a plant or a seed thereof the Pseudomonas protegens mutant strain CHA0-ΔretS-NiF, or a composition or a microbial agent comprising said strain.

The plants to which the present invention relates may be monocotyledonous or dicotyledonous plants, such as plants of cruciferae, Gramineae, liliaceae, and the like.

The mutant strain of Pseudomonas protegens CHA0 is CHA0-ΔretS-NiF. It is a Gram-stained negative strain having rod-shaped cells. The colonies are pale yellow and have nicked edges. It performs aerobic respiration and has the best growth status in KB medium at the optimal growth temperature of 28° C. Generally, after a period of KB culture, the fermentation broth is earthy yellow or light brick red, with a lot of foam. Compared to wild-type CHA0 bacteria, retS gene on the chromosome of the mutant strain CHA0-ΔretS-NiF is knocked out, and a NiF gene island with biological nitrogen-fixing function is inserted at the same time.

Its deposit number is CGMCC No. 14476 (Deposited by: China General Microbiological Culture Collection Center Institute of Microbiology, Address: NO. 1 West Beichen Road, Chaoyang District, Beijing 100101, China, Deposit date: Jul. 31, 2017).

The present invention also relates to a fermentation culture method of Pseudomonas protegens mutant strain CHA0-ΔretS-NiF, comprising the following steps:

(1) Seed activation: removing a glycerol tube containing the CHA0-ΔretS-NiF strain from a −80° C. ultra-low temperature freezer, after thawing, taking a small amount of bacterial solution and streaking it on a LB+genta20 plate, invertedly incubating it in an constant temperature biochemical incubator at 30° C. for 20 hours, and randomly selecting 5 single colonies from the plate to perform colony PCR verification to ensure that the correct target strain is obtained;

(2) Shake flask seed culture: inoculating the activated CHA0-ΔretS-NiF strain into KB medium and placing it in a full-temperature shaking incubator for 20 hours to obtain the seed solution;

(3) Fermenter culture: inoculating the seed solution into a fermenter containing KB medium at the inoculation amount of 5-10% (5-10 ml seed solution per 100 ml of KB medium), after the inoculation, setting aeration volume, dissolved oxygen, temperature, rotation speed and pH, taking the bacterial solution every 6 h to measure the cell density, wherein the fermentation period is 96 h.

As one of the preferred technical solutions, the formula of the KB medium in steps (2) and (3) is: 10 mL glycerol, 20 g peptone, 1.5 g K₂HPO₄, 1.5 g MgSO₄.7H₂O per 1000 mL of water.

As one of the preferred technical solutions, the conditions of the shake flask seed culture in step (2) are 30° C. and 200 rpm.

As one of the preferred technical solutions, the conditions of the fermenter culture in step (3) are: a temperature of 26 to 32° C., a pH of 6 to 7.5, a rotation speed of 300 to 600 rpm, a ventilation volume of 0.8 to 4.0 L/min, and dissolved oxygen of 0.8-1.0 L/min. Dissolved oxygen is not connected in series to the rotation speed. After culturing for 12-24 hours, 25-100 mL of 50 wt % glucose aqueous solution is added by flow. After that, 25-100 mL of 50% glucose aqueous solution is added by flow every 2 to 6 hours until the end of fermentation. During the period, 20 v/v % of phosphoric acid/ammonia is used to maintain a stable pH, and 50 v/v % of antifoaming agent is used for defoaming.

As one of the further preferred technical solutions, the conditions of the fermenter culture in step (3) are: the temperature is 28° C., the pH is 7, and the rotation speed is 600 rpm.

The invention also provides a microbial agent comprising Pseudomonas protegens CHA0 mutant strain CHA0-ΔretS-NiF as an active ingredient.

As one of the preferred technical solutions, the method for preparing the microbial agent is: in step (3), when the bacterial cells cultured in the fermenter enter a stable phase, and the cell density reaches the maximum, the cells are centrifuged, and the bacterial cells are collected after lyophilization.

The beneficial effects of the present invention:

The invention provides a fermentation method of Pseudomonas protegens mutant strain CHA0-ΔretS-NiF. The optimum growth conditions are a pH of 7, a temperature of 28° C., and a rotation speed of 600 rpm. According to the existing literature, there are few reports on the fermentation methods of Pseudomonas protegens. Therefore, the determination of the optimal growth conditions of the mutant strain CHA0-ΔretS-NiF is not only conducive to the expansion of the strain, but also to obtain a large number of bacterial cells. The possibility of industrial production is increased. It also provides reference for the fermentation culture of other Pseudomonas protegens in the same genus.

The invention provides Pseudomonas protegens mutant strain CHA0-ΔretS-NiF and a microbial agent using the same as an active ingredient. A room-temperature pot test proves that the strain can effectively promote plant growth. Mainly because the knockout of retS gene increases the yield of the secondary metabolite 2,4-DAPG in the mutant strain, the bactericidal ability is enhanced. In the meantime, the integration of the nitrogen-fixing gene cluster NiF makes the mutant strain have nitrogen-fixing ability. It provides a nitrogen source for the plant and satisfies the requirement of the plant for nitrogen sources during growth.

### DESCRIPTION OF THE DRAWINGS

FIG. 1 is a flowchart of knocking out retS gene on the chromosome of the CHA0 bacterium.

FIG. 2 is a diagram showing the colony PCR verification of the 25 Pseudomonas protegens mutant strain CHA0-ΔretS of the present invention.

FIG. 3 is a diagram showing the enzymatic identification of the expression plasmid pBeloBAC11-oriT-TnpA-genta-NiF constructed by the Red/ET direct cloning method of the present invention by restriction endonuclease Kpn I.

FIG. 4 is a flowchart of the constructing experiment of Pseudomonas protegens mutant strain CHA0-ΔretS-NiF of the present invention.

FIG. 5 is a diagram showing the colony PCR verification of the Pseudomonas protegens mutant strain CHA0-ΔretS-NiF of the present invention.

FIG. 6 shows the results of the bacteriostatic tests of Pseudomonas protegens of the present invention against Bacillus subtilis.

FIG. 7 is a chromatogram of the yield of the antibiotic 2,4-DAPG synthesized by the wild-type strain CHA0 and its mutant strain.

FIG. 8 shows the results of determination of the nitrogenase activities of the experimental strain Pseudomonas protegens CHA0 and its mutant strains CHA0-NiF, CHA0-ΔretS, and CHA0-ΔretS-NiF.

FIG. 9 shows the results of detecting the expression levels of key genes Nif D, Nif K, Nif N, Nif M, Nif Q, Nif S, Nif T in the genomes of engineered strains CHA0-ΔretS-NiF and Pseudomonas stutzeri DSM4166 by fluorescent quantitative PCR.

FIG. 10 shows the optimal pH for growth of the mutant strain CHA0-ΔretS-NiF.

FIG. 11 shows the optimal temperature for the growth of the mutant strain CHA0-ΔretS-NiF.

FIG. 12 shows the optimal rotation speed for the growth of the mutant strain CHA0-ΔretS-NiF.

FIG. 13 shows the growth of the mutant strain CHA0-ΔretS-NiF in a 5 L fermenter.

FIG. 14 shows the results of determination of nitrogenase activities of different transformants of the mutant strain CHA0-ΔretS-NiF.

FIG. 15A is a diagram showing the effect on Arabidopsis thaliana administered with different microbial agents after being transplanted into a pot for 4 weeks

FIG. 15B is a schematic diagram showing the diameter of the rosette of Arabidopsis thaliana administered with different microbial agents after being transplanted into a pot for 4 weeks.

FIG. 16 shows the biological traits of various experimental treatments during the late growth period of garlic. Among them, the leaves of treatment 1 are on the left, and the leaves of treatment 4 are on the right.

### DEPOSIT INFORMATION

Classification designation: Pseudomonas protegens mutant strain CHA0-ΔretS-NiF

Name of the depository: China General Microbiological Culture Collection Center Institute of Microbiology

Address of the depository: NO. 1 West Beichen Road, Chaoyang District, Beijing 100101, China

Deposit date: Jul. 31, 2017

Deposit number: CGMCC NO. 14476

### DETAILED DESCRIPTION

The present invention will be further described in conjunction with the drawings and the Examples. The following description is to explain the present invention and will not limit its contents.

The mutant strain of Pseudomonas protegens CHA0 is CHA0-ΔretS-NiF, and its accession number is CGMCC No. 14476 (Deposited by: China General Microbiological Culture Collection Center Institute of Microbiology, Address: NO. 1 West Beichen Road, Chaoyang District, Beijing 100101, China, Deposit date: Jul. 31, 2017).

### Example 1

A method for screening Pseudomonas protegens mutant strain CHA0-ΔretS, comprising the specific steps as follows:

(1) The plasmid pBBR1-Rha-TEGpsy-kan (which can express recombinases in Pseudomonas) was introduced into the wild type 30 Pseudomonas protegens CHA0 by electrotransformation. The electrotransformed bacteria were coated on a plate of LB medium (components of LB medium: tryptone 10 g/L, yeast extract 5 g/L, sodium chloride Ig/L, pH 7.0)+kanamycin (km, 30 μg/mL), and 12 single colonies were selected randomly to extract the plasmids to be enzymatically identified, and the correct transformant CHA0::pBBR1-Rha-TEGpsy-kan was screened;

(2) retS gene in the genome of Pseudomonas protegens CHA0 was knocked out. The linear DNA fragment loxM-genta (which was obtained by PCR method using a pair of primers, RetS-Genta-loxM-5′ GCACACGCCCTTGCCGTGCGGTCATTACGCCGCGCATAGTTATAA TCAGGCATCAACCAACGAAGGGATTTCGCCAGCTGAATTACATTC CCAACCG/RetS-Genta-loxM-3′TGGAGCATGGTGGGAGCTCACGAC TAAAGGAGGGCGAGCGAGAGTTTAACAGGCGCCGCAGAGCCTGT CGGCTCACAACTTAAATGTGAAAGTGGGTC, shown in SEQ ID NO.15 and SEQ ID NO.16 respectively) was electrotransformed into CHA0::pBBR1-Rha-TEGpsy-kan obtained in step (1). Using the method of Red/ET homologous recombination, under the action of the recombinase, retS gene in the genome of Pseudomonas protegens CHA0 was replaced by gentamicin resistance gene (genta). Multiple single colonies were selected randomly to be subject to PCR verification (the pair of primers used for verification are check-5′TGCTTCTACCGCAAGGACATC/check-3′GCTGATGAAGCACGAGAGCAC, shown in SEQ ID NO.13 and SEQ ID NO.14 respectively). The correct transformant CHA0::ΔretS-genta-loxM was screened.

(3) The genta resistance gene in CHA0::ΔretS-genta-loxM was eliminated. PCM157 plasmid capable of expressing Cre recombinase was electrotransformed into CHA0::ΔretS-genta-loxM, and was coated on a plate of LB medium+tetracycline (tet 25 μg/mL) for screening. The resultant recombinants were inoculated into 1 mL LB+tet 25 μg/mL liquid medium, and cultured at 900 rpm, 30° C. overnight. 50 μL of the overnight cultured bacterial solution was transferred to 1 mL of fresh LB+tet 25 μg/mL liquid medium. After culturing at 900 rpm for 3 hours at 30° C., 1 mM of isopropyl-β-D-thiogalactoside (IPTG) was added for induction. After continuing the culture for 2 hours, the bacterial solution was streaked in a Z-shaped line on a LB plate with a blue inoculation loop. After single colonies grew, they were double-streaked on a LB plate and a LB+genta 15 μg/mL plate respectively and cultured at 30° C. overnight. If single colonies grew on both plates, it indicated that the genta resistance gene in the recombinant had not been eliminated; If the colonies grew on LB plate while did not grow on the LB+genta 15 μg/m plate, it indicated that the genta resistance gene in the recombinant had been eliminated. Such recombinants whose genta resistance gene had been eliminated were picked up and subjected to colony PCR verification and sequencing, using the following primers:

check-5′ TGCTTCTACCGCAAGGACATC/
check-3′ GCTGATGAAGCACGAGAGCAC;
as shown in SEQ ID NO. 13 and
SEQ ID NO.14 respectively.

(4) The correct transformant CHA0-ΔretS was cryopreserved after PCR verification and sequencing for subsequent bacteriostatic, room temperature potting and field trials.

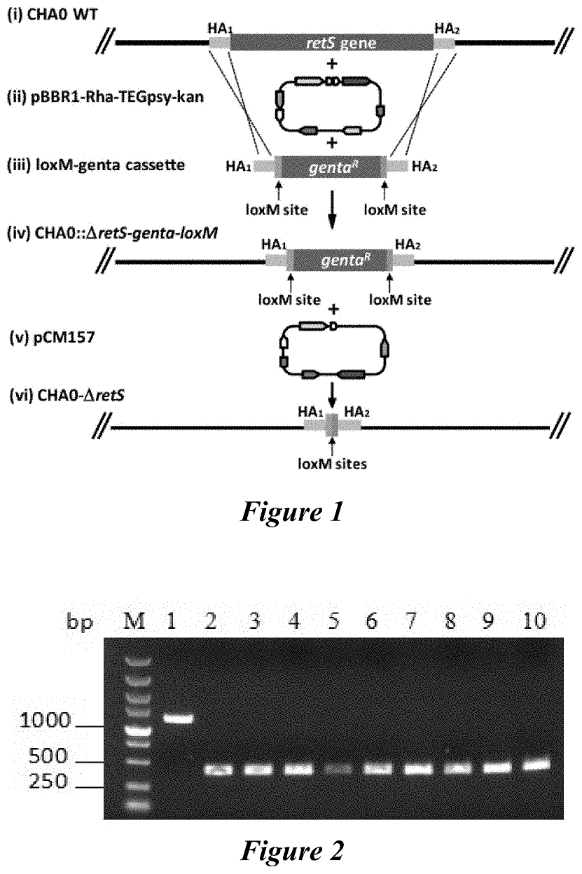

FIG. 1 is a flowchart of knocking out retS gene on the chromosome of the CHA0 bacterium. FIG. 2 is a diagram showing the colony PCR verification of the CHA0-ΔretS. As shown in the figure, M is the marker of DL 5,000 DNA, sample 1 is the wild type CHA0 as a control, and samples 2-10 are the final transformant CHA0-ΔretS. Under the action of the Cre recombinase introduced by IPTG, specific recombination between two loxM sites (sequences) was mediated, and the genta resistance gene sequence between the loxM sites was deleted. Therefore, the effects of the exogenous resistance gene on the growth, reproduction and colonization of Pseudomonas protegens CHA0 were eliminated. It made the strain safer to be used.

### Example 2

A method for screening Pseudomonas protegens mutant strain CHA0-ΔretS-NiF, comprising the specific steps as follows:

(1) Using Red/ET direct cloning method, the restriction endonucleases Afl II and Ssp I were used to digest the genomic DNA of Pseudomonas stutzeri DSM4166 to obtain a 49 kb NiF nitrogen-fixing gene island, which was verified by DNA fragment gel electrophoresis, and ligated to the corresponding vector. The primers used were:

Primer 1:
AGTGAATTGTAATACGACTCACTATAGGGCGAATTCGAGCTCGGTACCCGC
TTAAGTACGGCTACCTGGAGCTCGCGCCAGTG,
as shown in SEQ ID NO.
Primer 2:
TACGGCTACCTGGAGCTCGCGCCAGTGCTTGCCGACATCGAATCACGGCCG
CTGCTGCAGCACGTGGTGGTCACCGGCCGGGATCCGTTTAAACACAAATGG
CAAGGGCTAATG,
as shown in SEQ ID NO. 2;
Primer 3:
ATTGATGTTTTCCTTGGCCAGCGCCTCGAACATCCGGCTGGCGACGCCTGC
GTGCGAACGCATACCGACACCGACGATAGGGATCCGTTTAAACGGTGTGGT
AGCTCGCGTATT,
as shown in SEQ ID NO. 3;
Primer 4:
GCGACACTATAGAATACTCAAGCTTGGCATGAATGCAGGTCGACTCTAGAG
AATATTGATGTTTTCCTTGGCCAGCGCCTCGAAC,
as shown in SEQ ID NO. 4.

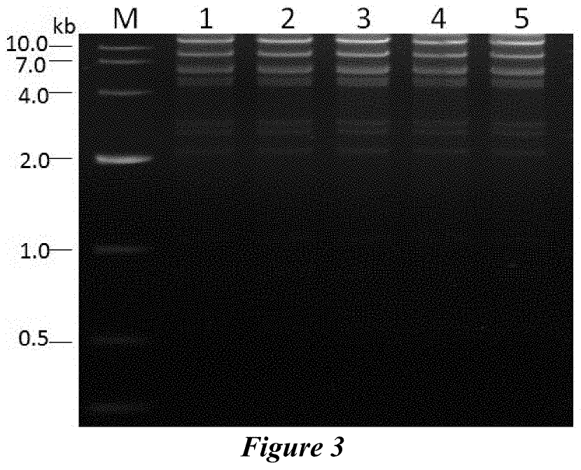

The expression plasmid pBeloBAC11-oriT-TnpA-genta-NiF (FIG. 3) was constructed and was identified by digesting with restriction endonuclease Kpn I. The correct plasmid was electrotransformed into E. coli ET12567.

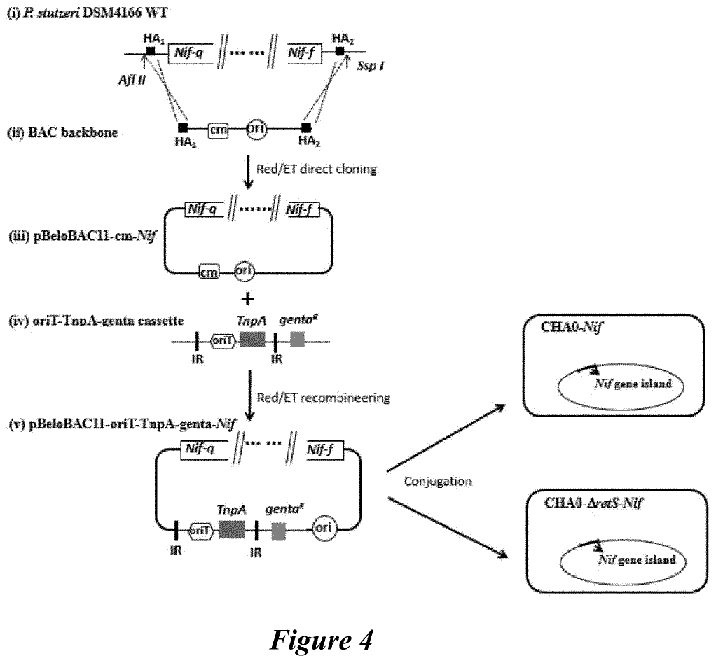

(2) The plasmid pBeloBAC11-oriT-TnpA-genta-NiF from E. coli ET12567 was introduced into Pseudomonas protegens CHA0-ΔretS by conjugative transfer, and then NiF gene was randomly inserted into the genomic DNA of CHA0 by transposition (FIG. 4). The detailed operation of the conjugation transfer was as follows: A single colony of Pseudomonas protegens CHA0-ΔretS was picked up and was cultured (LB medium, 30° C.) separately with E. coli ET12567 (LB+genta 2 μg/mL+cm 10 μg/mL+km 1 μg/mL medium, 37° C.) overnight; The two overnight bacterial solutions were centrifuged at 7000 rpm for 1 minute. Pseudomonas protegens CHA0-ΔretS and E. coli ET12567 were washed twice with fresh LB medium, resuspended in 300 μL of LB medium. 50 μL of each suspension was mixed and coated on a small area in the middle of the LB plate and air dried. After incubating for 4 hours at 37° C., the plate was invertedly incubated in an incubator at 30° C. overnight; The bacteria on the plate were scraped with an inoculating loop, mixed thoroughly with 1 mL sterilized solution. 100 μL of bacterial solution was streaked in a Z-shaped line on a plate of PMM medium (8 g/L dipotassium phosphate, 5 g/L potassium dihydrogen phosphate, Ig/L ammonium sulfate, 6.6 g/L sodium succinate, pH adjusted to 7.0, 1.2 mL/L of 1M magnesium sulfate added after sterilization)+genta 25 μg/mL, and cultured invertedly at 30° C. for 2 days until single colonies appeared; Two days later, colonies grew. A single colony was picked up to be inoculated in 1 mL of LB+genta 25 μg/mL and to be cultured overnight, followed by colony PCR verification using the following 5 pairs of primers:

NiF-check-1 GGTCTACCAGCTCGACCT/
NiF-check-2 CGATTCCAGCGTCGAATGAT;
NiF-check-3 GCTGACCTCCTTGAGGTGCT/
NiF-check-4 CAGCGGCACCTCGAGGAGT;
NiF-check-5 GATAGAGCAGGTCCTCGAT/
NiF-check-6 GGTGCTCTACGTCAGCCATT;
NiF-check-7 CGACAGATCCTGATTACCGT/
NiF-check-8 TACCCTCGACCAGCTTGAGCA;
check-5′ TGCTTCTACCGCAAGGACATC/
check-3′ GCTGATGAAGCACGAGAGCAC;
as shown in SEQ ID NO. 5 to
SEQ ID NO. 14, respectively.

The first four pairs of primers were used to verify whether the NiF nitrogen-fixing gene had been integrated as a whole into the genome of Pseudomonas protegens CHA0-ΔretS. The amplified PCR fragments were 1000 bp, 970 bp, 830 bp, and 1080 bp, respectively. The fifth pair of primers was used to verify that the strain to which the NiF nitrogen-fixing gene was introduced was Pseudomonas protegens CHA0 instead of Escherichia coli ET12567, and the PCR amplification result was retS gene with a DNA fragment size of 3200 bp.

(3) The correct transformant CHA0-ΔretS-NiF was sent to the sequencing after colony PCR verification, and that with the correct results was cryopreserved and used for subsequent bacteriostatic, room temperature potting and field trials.

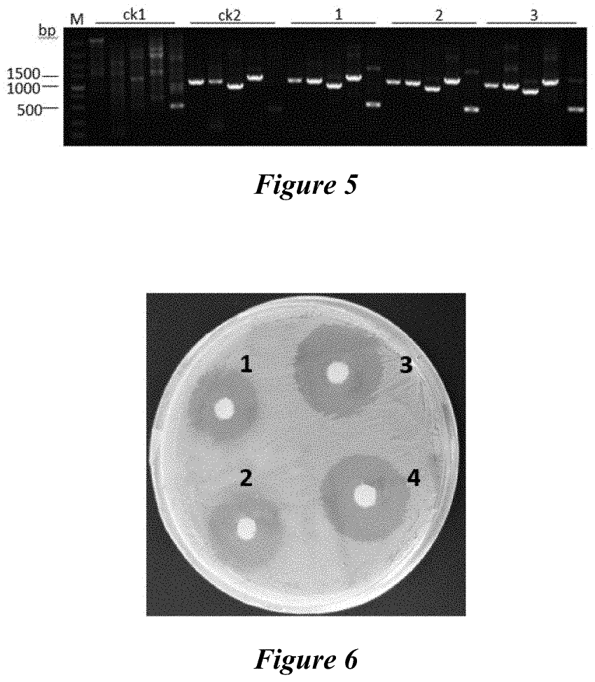

FIG. 5 shows that M is the marker of DL 5,000 DNA. ck1 is the mutant Pseudomonas protegens CHA0-ΔretS in which retS gene has been knocked out and ck2 is E. coli ET12567, which two serve as control groups. Samples 1, 2 and 3 were CHA0-ΔretS-NiF transformants randomly selected from the plate, and each was subjected to 5 colony PCR verifications with the above 5 pairs of primers respectively. After repeated careful comparison, it was found that the data obtained were consistent with the expected results. Thus, it was proved that the NiF nitrogen-fixing gene in Pseudomonas stutzeri DSM4166 had been integrated into the genome of Pseudomonas protegens CHA0-ΔretS as a whole, and the correct transformant was obtained.

The information about Pseudomonas protegens CHA0 and its mutant strains CHA0-NiF, CHA0-ΔretS and CHA0-ΔretS-NiF obtained by the present invention is shown in Table 1.

TABLE 1
Information about each strain

| Strains of | | |
|---|---|---|
| Pseudomonas | | |
| protegens | Relevant properties | origins |
| CHA0 | Wild type | German Collection of |
| | | Microorganisms, DSMZ |
| CHA0-NiF | CHA0 genetically engineered strain having | The present invention |
| | integrated NiF nitrogen-fixing gene, having genta | |
| | resistance during culture, and capable of | |
| | biologically nitrogen-fixing and reducing the usage | |
| | of nitrogen fertilizer during the growth of plants | |
| CHA0-ΔretS | CHA0 mutant strain whose retS gene has been | The present invention |
| | knocked out, having no genta resistance gene, | |
| | having no resistance during culture, and having | |
| | increased bacteria killing activity | |
| CHA0-ΔretS-NiF | CHA0 genetically engineered strain whose retS gene | The present invention, |
| | has been knocked out and having integrated NiF | deposited |
| | nitrogen-fixing gene, having genta resistance during | |
| | culture, having increased bacteria killing activity, | |
| | and capable of biologically nitrogen-fixing and | |
| | reducing the usage of nitrogen fertilizer during the | |
| | growth of plants | |

### Example 3

The filter paper method was used to detect the inhibitory effects of Pseudomonas protegens CHA0 and its mutant strains CHA0-NiF, CHA0-ΔretS and CHA0-ΔretS-NiF on Bacillus subtilis. The specific steps were as follows:

(1) Bacillus subtilis and the experimental strain Pseudomonas protegens CHA0 and its mutant strains CHA0-NiF, CHA0-ΔretS and CHA0-ΔretS-NiF were inoculated into 1 mL LB liquid medium respectively, and were cultured at 900 rpm, overnight at 30° C.;

(2) The next day, Bacillus subtilis was centrifuged at 9000 rpm for 1 minute, and 100 μL of the bacterial solution was uniformly coated on a LB solid medium (15 g/L agar was added on the basis of liquid LB medium) plate. After dried, several double-layer filter paper sheets having a diameter of 6 mm were placed on the plate. 5 μL overnight cultured experimental strain Pseudomonas protegens CHA0 and its mutant strains CHA0-NiF, CHA0-ΔretS and CHA0-ΔretS-NiF were added dropwise to the filter paper sheets. The plate was cultured at 30° C. overnight.

(3) On the third day, the size of the inhibition zone around each small filter paper sheet on the plate was observed.

FIG. 6 shows that the inhibition zones of the Pseudomonas protegens CHA0 mutant strains \textcircled{3} CHA0-ΔretS and \textcircled{4} CHA-ΔretS-NiF whose retS gene had been knocked out were much larger than those of Pseudomonas protegens \textcircled{1} CHA0 and \textcircled{2} CHA0-NiF whose retS gene had not been knocked out (the diameter of the inhibition zones of sample \textcircled{3} and \textcircled{4} is 2.7 cm, and that of samples \textcircled{1} and \textcircled{2} is 2.3 cm). It indicated that the ability for inhibiting Bacillus subtilis was increased after ΔretS gene was knocked out.

### Example 4

High-performance liquid chromatography analysis of increased antibiotic 2,4-DAPG production led by retS gene knockout

The experimental strains, Pseudomonas protegens CHA0 and its mutant strains CHA0-NiF, CHA0-ΔretS and CHA0-ΔretS-NiF, were cultured in KB medium at 150 rpm and 30° C. for 24 hours, and then 1 mL of resin was added. After the bacterial solution with the resin was shook for 24 h, it was centrifuged at 8000 rpm for 10 min to collect the resin, and an equal volume of ethyl acetate was added for overnight extraction. The supernatant was centrifuged again and was spin-evaporated to obtain an extract, which was reconstituted by adding 1 mL of methanol for HPLC detection. Conditions for HPLC detection were: Thermo Scientific Acclaim™18 reverse-phase column (2.1 mm×100 mm, 2.2 μm), column temperature 30° C.; mobile phase: composed of 0.1% acetic acid aqueous solution (solvent A) and acetonitrile (solvent B); chromatography program: 0-5 min, 5% solvent B; 5-20 min, 5%-95% solvent B; 20-25 min, 95% solvent B; flow rate was 0.5 mL/min. Ultraviolet (UV) light (2,4-DAPG, λ=270 nm) was monitored at 250 nm, 270 nm, 290 nm, and 310 nm, respectively. MS measurement was performed on an amaZon velocity mass spectrometer and ultra-high resolution Qq-Time-Of-Flight using a standard ESI (electrospray ionization) source.

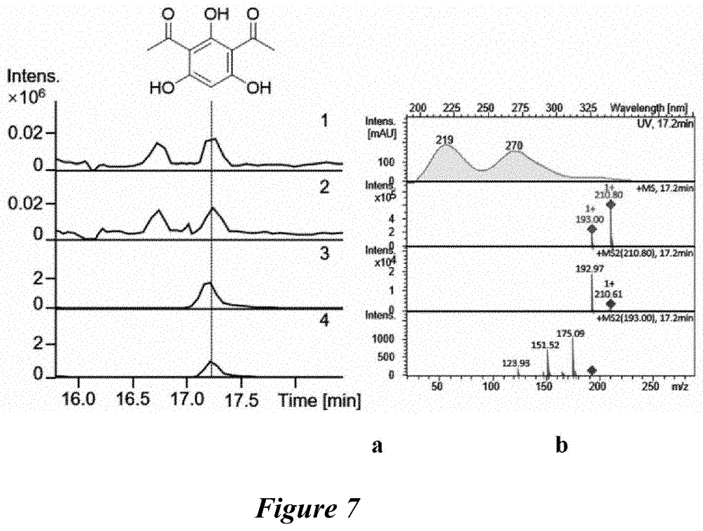

FIG. 7a shows 1, WT CHA0; 2, CHA0::NiF; 3, CHA0-ΔretS; 4, CHA0-ΔretS-NiF. The results showed that the yields of 2,4-DAPG of the engineered strains in which retS gene was knocked out were much higher than strains without retS knockout. The yields of the antibiotic 2,4-DAPG were increased by about 100 times. FIG. 7b shows the ultraviolet absorption peak and mass spectrum of antibiotic 2,4-DAPG.

### Example 5

Determination of Nitrogenase Activity by Acetylene Reduction

(1) The experimental strains, Pseudomonas protegens CHA0 and its mutant strains CHA0-NiF, CHA0-ΔretS and CHA0-ΔretS-NiF, were respectively inoculated into LB medium and cultured at 30° C. for 8 hours.

After centrifugation at 5000 rpm for 10 min at 4° C., the bacterial solution was collected and washed three times with 0.85% physiological saline, and was resuspended with a nitrogen-free medium (glucose 10 g/L, potassium dihydrogen phosphate 0.2 g/L, magnesium sulfate heptahydrate 0.2 g/L, sodium chloride 0.2 g/L, calcium sulfate dihydrate 0.2 g/L, calcium carbonate 5 g/L, adjusted to pH 7.0-7.2, sterilized at 113° C. for 30 min) to OD=1.0.

(3) 18 mL of nitrogen-free medium and 2 mL of the above bacterial solution were added to a 100 mL anaerobic culture bottle. Air was removed and replaced with the high-purity argon to make the anaerobic bottle sealed. After adding 1% oxygen, and incubation at 30° C., 250 rpm for 6 h, 10% mixed gas was extracted and 10% acetylene gas was injected. After incubation at 30° C., 250 rpm for 4 h, it was sampled for determination. 100 μL of mixed gas was taken from the bottle with a sterile syringe for sampling, and injected into a gas chromatograph for determination of ethylene content. An anaerobic bottle without bacterial injection was used as a control.

(4) Nitrogenase activity=detected ethylene peak area×(anaerobic flask volume-sample volume)/(standardized ethylene gas peak area×reaction time×protein concentration).

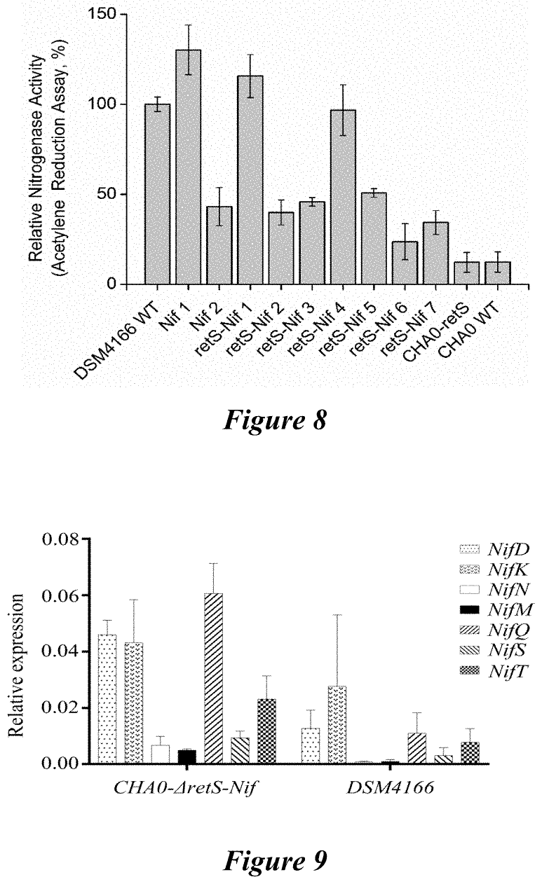

The results in FIG. 8 showed that Pseudomonas protegens CHA0 mutant strains in which the nitrogen-fixing gene island (NiF) was integrated all had nitrogenase activity, but the expression levels were different. Among them, the mutant strain retS-NiF1 had the highest nitrogenase activity. The strain was stored frozen for subsequent fermentation experiments and field experiments.

### Example 6

Quantitative real-time PCR was used to detect the expression of related key genes in the nitrogen-fixing gene island (NiF) in CHA0-ΔretS-NiF and Pseudomonas stutzeri DSM4166.

The engineered strains CHA0-ΔretS-NiF and Pseudomonas stutzeri DSM4166 were respectively cultured in KB medium for 24 hours, moved to nitrogen-free medium for 6 hours, and the total RNA of the target strains was extracted using the RNAPure kit. Then, genomic DNA was removed from RNA by reverse transcription kit (purchased from Takara, Japan) to synthesize complementary DNA strands (cDNA). GAPDH gene was used as an internal reference gene to make fluorescence quantitative analysis (SYBR® Premix Ex Taq™ II (Tli RNaseH Plus) Code No. RR820A) for the expression levels of seven key genes NiFD, NiFK, NiFN, NFM, NiFQ, NiFS, NiFT in the nitrogen-fixing gene island (NiF). FIG. 9 showed that these seven key genes had certain relative expression levels in the engineered strain CHA0-ΔretS-NiF, and all of them were higher than the expression levels in Pseudomonas stutzeri DSM4166. Thus it confirmed that nitrogenase was produced in Pseudomonas protegens CHA0.

### Example 7

Fermentation method of Pseudomonas protegens CHA0 mutant strain CHA0-ΔretS-NiF

(1) Seed activation: A glycerol tube was removed from a −80° C. ultra-low temperature freezer. After thawing, a small amount of bacterial solution was dipped by a 1 ul inoculation ring to streak on a LB+genta 20 plate. After invertedly placed in a biochemical incubator and incubated at 30° C. for 20 h at the constant temperature, 5 single colonies were randomly selected from the plate to perform colony PCR verification to ensure that the correct target strain is obtained;

(2) Shake flask seed culture: A small amount of bacteria on the LB+genta 20 plate in which the seed is activated was scraped with a 1 uL inoculation loop, and was inoculated into the KB medium. 100 mL KB medium was added into a 500 mL bottle, and was incubated in a full-temperature shaking at 30° C., 200 rpm for 20 hours. The components of the KB medium were 10 mL glycerol, 20 g peptone, K₂HPO₄ 1.5 g, MgSO₄.7H₂O 1.5 g per 1000 mL water.

(3) Fermenter culture: After the seed solution in the shake flask was successfully cultured, the seed solution was sucked into a 60 ml syringe in a clean bench, and then the bacterial solution was injected into a 1 L fermenter from the inoculation port by puncture method. The inoculation amount was 5% of the total volume of liquid KB medium contained in the fermenter. After the inoculation, the aeration volume, dissolved oxygen, temperature, rotation speed, and pH were intelligently set as follows: aeration volume 0.8 L/min, dissolved oxygen 0.8 L/min, dissolved oxygen was not connected in series with the rotation speed. The bacterial solution was taken every 6 h to determine the cell density. The fermentation period was 96 h. After 24 h of culture, 25 mL of 50% glucose aqueous solution was added by flow, and then 25 mL of 50% glucose aqueous solution was added by flow every 6 h until the end of fermentation. During the period, 20 v/v % of phosphoric acid/ammonia was used to maintain a stable pH, and 50 v/v % of antifoaming agent was used for defoaming

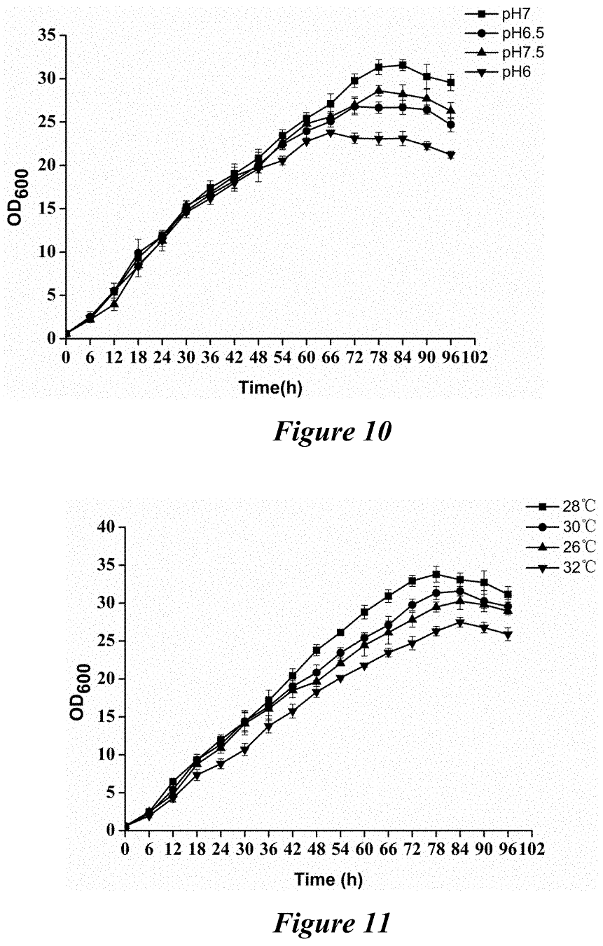

CHA0-ΔretS-NiF strain was cultured when pH in the above step (3) was set to 6, 6.5, 7 and 7.5 respectively while other conditions and steps were not changed. The growths of CHA0-ΔretS-NiF strain under different culturing conditions were compared and the results were showed in FIG. 10.

From FIG. 10, it can be seen that during the 48 h culture period, growth environments at different pH did not have a large effect on the density of the bacteria. The growth course remained substantially the same. After 48 h, however, probably due to the fact that the cell density reached a certain level and metabolites that were not conducive to the growth of the bacteria were secreted to a concentration that reached the cell's highest ability to withstand, the amount of the cells were not high in an acidic or alkaline environment.

The CHA0-ΔretS-NiF strain was cultured when the temperature in step (3) above were set to 26° C., 30° C., and 32° C. respectively, and the remaining conditions and steps were unchanged. The growths of the CHA0-ΔretS-NiF strain under different temperature conditions were compared, and the results were shown in FIG. 11.

It can be seen from FIG. 11 that 28° C. was the optimum temperature for the growth of the engineered strain CHA0-ΔretS-NiF. The target strain under the 32° C. culture conditions showed a poor growth state from the beginning, but after 36 h, under the 28° C., 30° C., and 26° C. conditions, the increase of cells began to change. Finally, the advantage of the growth of the cells at 28° C. was relatively obvious. This may be related to the regulation of the metabolic pathways of microorganisms by temperature, which affected the reaction rate of enzymes.

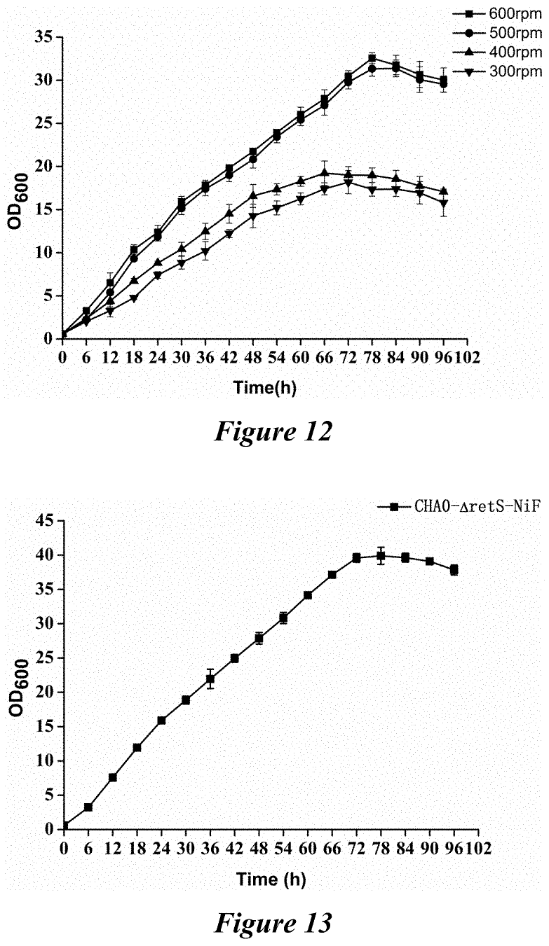

The CHA0-ΔretS-NiF strain was cultured when the rotation speed in step (3) above was set to 300 rmp, 400 rmp, and 500 rmp respectively, and the remaining conditions and steps were unchanged. The growths of the strain under different rotation speed conditions were compared, and the results were shown in FIG. 12.

It can be seen from FIG. 12 that 600 rpm was the optimum growth rotation speed of the bacterium, but the growth advantage of 600 rpm over 500 rpm was not very obvious, which indicated that the 500 rpm condition could basically meet the oxygen demand during the growth of the bacterial cells. The amount of dissolved oxygen could affect the metabolic pathways of microorganisms, product yield, enzyme activity, and appropriate oxygen concentration was conducive to the growth of bacterial cells, but excessively high oxygen concentration would accelerate the oxidation of cells and cause the bacterial cells to enter the decline stage in advance.

As described above, the optimal environmental growth condition of the engineering strain CHA0-ΔretS-NiF of the present invention was the pH of 7, the temperature of 28° C., and the rotation speed of 600 rpm.

### Example 8

5 L fermenter expansion culture of CHA0-ΔretS-NiF under optimal environmental growth conditions and collection of bacterial cells

(1) Seed activation: A glycerol tube was removed from a −80° C. ultra-low temperature freezer. After thawing, a small amount of bacterial solution was dipped by a 1 ul inoculation ring to streak on a LB+genta 20 plate. The plate was invertedly placed in a biochemical incubator and incubated at 30° C. for 20 h;

(2) Shake flask seed culture: A small amount of bacteria on the LB+genta 20 plate in which the seed is activated was scraped with a 1 uL inoculation loop, and was inoculated into the KB medium. 100 mL KB medium was added into a 500 mL bottle, and was incubated in a full-temperature shaking at 30° C., 200 rpm for 20 hours. The components of the KB medium were 10 mL glycerol, 20 g peptone, K₂HPO₄ 1.5 g, MgSO₄.7H₂O 1.5 g per 1000 mL water.

(3) Fermenter culture: Cotton soaked with alcohol was wrapped near the inoculation port of a 5 L fermenter and ignited. After the flame surrounded the inoculation port, the screw at the inoculation port was unscrewed with tweezers. At this time, the bacterial solution in the shake bottle was quickly poured into the fermenter. Before the flame went out, the screw was quickly screwed back to the inoculation port. The inoculation amount was 10% of the total volume of the liquid KB medium in the fermenter. After the inoculation, the aeration volume, dissolved oxygen, temperature, rotation speed, and pH were intelligently set (temperature of 28° C., pH=7, rotation speed of 600 rpm, aeration volume 4 L/min, dissolved oxygen 1.0 L/min, dissolved oxygen was not connected in series with the rotation speed). The bacterial solution was taken every 6 h to determine the cell density. The fermentation period was 96 h. After 12 h of culture, 100 mL of 50% glucose aqueous solution was added, and then 50 mL of 50% glucose aqueous solution was added by flow every 2 h until the end of fermentation. During the period, 20 v/v % of phosphoric acid/ammonia was used to maintain a stable pH, and 50% of antifoaming agent was used for defoaming.

(4) When the bacterial cells entered a stable phase, and the cell density reached the maximum, the cells were centrifuged, and the bacterial cells were collected after lyophilization.

It can be seen from FIG. 13 that compared with the 1 L fermenter, the growth rate of the bacterial cells in the 5 L fermenter increased significantly in the logarithmic phase, and the final cell density also increased. The OD value was 40.76. The growth trends in the logarithmic phase were generally the same. That is, the growth rate in the early logarithmic stage was better than that in the middle and late logarithmic stages. The main reasons were: the aeration volume and rotation speed of the 5 L fermenter were better than those of the 1 L fermenter, and the stirring of the fan blades during the cultivation process could disperse the bacterial cells, avoid clumping, increase the contact area between the bacterial cells and the culture medium, and facilitate the absorption of oxygen.

After the fermentation was completed, the fermentation product was centrifuged, and the centrifuged bacterial cells were lyophilized. The dry weight of the bacterial cells was 15.9 g (DCW/L).

### Example 9

Use of Pseudomonas protegens mutant strain CHA0-ΔretS-NiF in killing bacteria, fixing nitrogen and promoting plant growth

1. The acetylene reduction method was used to determine the nitrogenase activities of different transformants of the mutant strain CHA0-ΔretS-NiF, and the mutant strain CHA0-ΔretS-NiF with the highest nitrogenase activity was selected from different transformants for subsequent pot experiments:

(1) The designated experimental strains stored at −80° C. were inoculated in LB liquid medium, and cultured at 30° C. for 8 h;

(2) After centrifugation at 5000 rpm for 10 min at 4° C., the bacterial solution was collected, washed three times with 0.85% physiological saline, and resuspended in a nitrogen-free medium to OD=1.0;

(3) 18 mL of nitrogen-free medium and 2 mL of the above bacterial solution were added in a 100 mL anaerobic culture bottle. Air was removed and replaced with high-purity argon to make the anaerobic bottle sealed. After 1% oxygen was added, and incubated a 30° C., 250 rpm for 6 hours, 10% of the mixed gas was extracted and 10% of acetylene gas was injected. After incubated at 30° C., 250 rpm for 4 hours, the sample was taken to be determined. The sampling was done by taking 100 μL of the mixed gas from the bottle with a sterile syringe and injecting it into a gas chromatograph for determining the content of ethylene. An anaerobic bottle injected with acetylene without bacterial inoculation was used as a control.

(4) Nitrogenase activity=Difference of the area of detected ethylene peaks×(volume of gas phase in the triangular flask/the amount of entered sample)/(area of standard ethylene gas peak×reaction time×protein concentration).

Protein concentration was determined according to the method of Coomassie Blue:

After Centrifuging 20 mL of bacterial solution at 4000 rpm for 10 min, the supernatant was discarded;

\textcircled{2} The pellet was resuspended with 200 μL 0.5M NaOH. After boiling for 5 min, 200 μL 0.5 M HCl was added, and the solution was centrifuged at 12000 rpm for 10 min.

\textcircled{3} 100 μL of the supernatant was taken, and 900 μL G250 was added to be mixed well. After the solution was placed for 5 minutes, the protein content was determined at OD595.

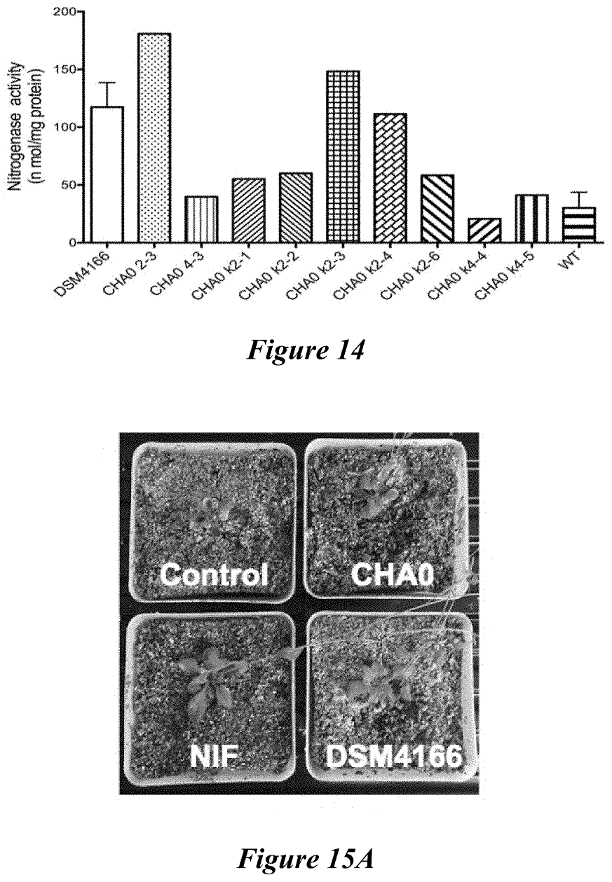

The results of measuring nitrogenase activities of different transformants of the mutant strain CHA0-ΔretS-NiF were shown in FIG. 14. Because the nitrogen-fixing gene island NiF was randomly inserted into the chromosome of the CHA0 bacteria, the levels of nitrogenase expressed by different transformants were also different. The results showed that the transformant CHA0 2-3 expressed the highest nitrogenase level, even higher than that of the wild-type strain DSM4166 carrying NiF.

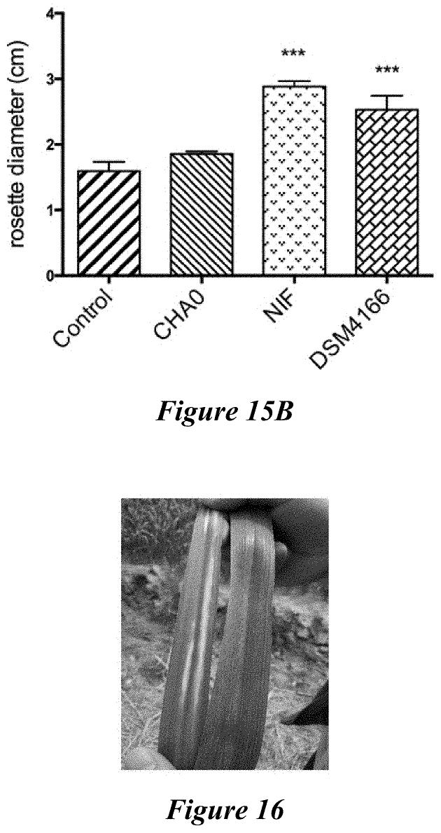

2. The wild-type Arabidopsis thaliana Col-0 was used as test object. The test conditions were temperature at 20° C., light intensity of 80 μmol·m⁻²·s⁻¹, light cycle: 16 hours light, 8 hours dark. The test was divided into 4 groups: The plant to which no nitrogen fertilizer was administered and bacterial solution of the wild-type Pseudomonas protegens CHA0 was administered was used as the negative control. The plant to which the bacterial solution of the original strain DSM4166 containing nitrogen-fixing gene cluster was administered was used as the positive control. The plant to which the bacterial solution of the preferred mutant strain CHA0 2-3 in step 1 which has the highest nitrogenase activity was administered was used as the test group. The effects of potting were shown in FIG. 15A.

3. The diameters of the rosettes of Arabidopsis thaliana were measured to compare the growth status of Arabidopsis thaliana. The results of measuring the diameters of the rosettes were shown in FIG. 15B.

It can be seen from FIG. 15A and FIG. 15B that in the Arabidopsis potting test, the plant to which no nitrogen fertilizer was administered and the wild-type Pseudomonas protegens CHA0 was administered was used as the negative control (Control and CHA0). The plant to which the original strain DSM4166 containing nitrogen-fixing gene cluster was administered was used as the positive control (DSM4166). The plant to which the bacterial solution of CHA0-ΔretS-NiF was administered was used as the test group (NIF). The results showed that the growth of Arabidopsis thaliana to which no nitrogen fertilizer was administered and the wild-type Pseudomonas protegens CHA0 was administered was not good, and the leaflets were small and the stem was short (FIG. 15A). In the positive control group to which no nitrogen fertilizer was administered, but to which the original strain DSM4166 containing nitrogen-fixing gene clusters was administered, and the test group to which the nitrogen-fixing Pseudomonas protegens CHA0-ΔretS-NiF was administered, the diameter of the rosette and the leaves of the Arabidopsis thaliana were better than those of the negative control. In addition, the diameter of the rosette and leaves of the Arabidopsis thaliana to which nitrogen-fixing Pseudomonas protegens CHA0-ΔretS-NiF was administered were better than those of the original strain DSM4166 containing nitrogen-fixing gene clusters (FIG. 15B).

### Example 10

Report of Field Test on Garlic with CHA0 Engineered Strains

1. Test time: October 2017-June 2018

2. Test location: Qianjiang Village, Yucheng Town, Yutai County, Jining City, Shandong Province

3. Test crop: hybrid garlic (white garlic)

4. Test treatments: The test was divided into 6 treatments as follows:

Treatment 1: Fertilizer was applied according to the farmers' convention (N 45 kg/hm², P₂O₅ 22.5 kg/hm², K₂O 22.5 kg/hm², organic fertilizer 40 kg/mu, high-nitrogen and high-potassium compound fertilizer for topdressing);

Treatment 2: Optimized fertilization, N 30 kg/hm², P₂O₅16 kg/hm², K₂O 24 kg/hm², N 30 kg/hm², P₂O₅ 16 kg/hm², K₂O 24 kg/hm², bio-organic fertilizer 200 kg/hm², formula fertilizer used for topdressing (18-5-17 humic acid type) 20 kg/mu; Dodine was used for seed dressing; hymexazol, methyl thiophanate and dodine were applied according to the actual situation in the spring when topdressing;

Chemical control measures (according to actual re-selection): mepiquat and brassinolide

Treatment 3: Microbial agent—Pseudomonas protegens Yun (purchased from Shandong Tainuo Pharmaceutical Co., Ltd.)

Fertilization was consistent with optimized fertilization. Pseudomonas protegens was used for seed dressing. Solution of Pseudomonas protegens was flushed by water to be applied during sowing, just before winter, during greening season, and during early spring. Bacterial solution was flushed by water to be applied during topdressing.

Treatment 4: Microbial agent—Pseudomonas protegens CHA0-ΔretS-NiF

Fertilization was consistent with optimized treatment. Pseudomonas protegens was used for seed dressing. Solution of Pseudomonas protegens was flushed by water to be applied during sowing, just before winter, during greening season, and during early spring. Bacterial solution was flushed by water to be applied during topdressing.

Treatment 5: Microbial agent—Pseudomonas protegens CHA0-ΔretS-NiF

The application amount of nitrogen fertilizer was ⅔ of optimized fertilization, and phosphorus and potassium were same as optimized treatment. Others were consistent with optimized treatment. Pseudomonas protegens was used for seed dressing. Solution of Pseudomonas protegens was flushed by water to be applied during sowing, just before winter, during greening season, and during early spring. Bacterial solution was flushed by water to be applied during topdressing.

The dosage form of the microbial agents was liquid, and the effective viable bacterial count was ≥1 billion/ml. The application amount was 2 kg/mu.

On Jan. 21, 2018, the length and width of the garlic leaves, the diameter of the stem, and the enzymatic activity of the root were measured before wintering (Table 1).

TABLE 1
Statistical table of biological traits of the test treatments (January 21)

| | item | | | | | |
|---|---|---|---|---|---|---|
| | | | | | | Enzymatic |
| | | | | | | activity |
| | | | | | | of root |
| | Length | Diameter | Width | Weight of | Weight of | mg/g |
| | of leaf | of stem | of leaf | seedling | root | (fresh weight |
| treatment | (cm) | (cm) | (cm) | (g) | (g) | of root)/h |
| treatment 1 | 20.8 | 11.61 | 0.978 | 107.25 | 16.15 | 0.335 |
| treatment 2 | 23.3 | 12.57 | 1.002 | 132.71 | 19.82 | 0.372 |
| treatment 3 | 24.1 | 13.02 | 1.062 | 137.26 | 21.54 | 0.410 |
| treatment 4 | 25.7 | 14.46 | 1.127 | 145.52 | 23.84 | 0.464 |
| treatment 5 | 25.3 | 13.98 | 1.123 | 144.86 | 23.01 | 0.441 |

TABLE 2
Statistical table of biological traits of product
demonstration test treatments (March 29)

item

| | Height | Diameter | Length | Width |
|---|---|---|---|---|
| | of plant | of stem | of leaf | of leaf |
| | (cm) | (cm) | (cm) | (cm) |
| treatment 1 | 772.2 | 18.64 | 403 | 37.3 |
| treatment 2 | 780.4 | 19.18 | 428 | 38.9 |
| treatment 3 | 794.2 | 19.35 | 453 | 39.9 |
| treatment 4 | 806.1 | 20.04 | 464 | 41.1 |
| treatment 5 | 798.4 | 19.63 | 450 | 40.3 |

The data in the above two tables were the growth indicators of the garlic measured at the seedling stage and the greening stage after winter. The measured data were consistent with the results of field test observations. The three treatments with Pseudomonas protegens in the early stage (seedling stage) had significant promotion effects on the width of leaf and the enzymatic activity of root of garlic. The most obvious promotion was treatment 4, which reached 38.5%. The reason was that the Pseudomonas protegens agents replaced the seed dressing with donine. Donine inhibited the growth of pathogens and also harmed the beneficial bacteria around the garlic so that indirectly hindered the growth of garlic in the early stage of growth. Therefore, the Pseudomonas protegens treated garlic seedlings grew vigorously.

In the early growth stages of garlic, the leaves were significantly longer and wider. Compared with control treatment 1, the lengths of leaves of treatments 4 and 5 were increased by 23.6% and 21.6%, respectively, and the widths of leaves were increased by 15.2% and 14.8%, respectively. This was consistent with the actual observations of treatments 4 and 5 in the field. The leaves of these two treatments were more stretched and grew better than those of control treatment 1.

The promotion effect on width of leaf in the late growth stage (greening stage after winter) of garlic was still maintained, and the diameters of the stems of garlic of the two Pseudomonas protegens treated groups were significantly larger than those of the control. Compared with control treatment 1, the lengths of leaves of treatments 4 and 5 were increased by 15.1% and 11.7%, respectively, and the widths of leaves were increased by 10.2% and 8.1%, respectively (FIG. 16).

From the planting of garlic in October to the harvest in May of the following year, the production was calculated and converted into the yield of garlic, and the results were statistically analyzed.

The garlic yield results of test treatments 1-5 are shown in Table 4 and 5.

TABLE 4
Differences in yield of garlic of different treatments

| | | | | | | Percent |
|---|---|---|---|---|---|---|
| | Test | Test | Test | | Increase | increase |
| | yield 1 | yield 2 | yield 3 | Yield/mu | of yield | of yield |
| treatment | (kg/mu) | (kg/mu) | (kg/mu) | (kg/mu) | (kg/mu) | (%) |
| treatment 1 | 1682.84 | 1748.21 | 1727.53 | 1719.53 | | |
| treatment 2 | 1779.56 | 1819.58 | 1788.89 | 1796.01 | 77.21 | 4.49 |
| treatment 3 | 1834.92 | 1921.62 | 1874.94 | 1877.16 | 158.36 | 9.21 |
| treatment 4 | 2008.29 | 2073.94 | 2069.39 | 2050.54 | 331.01 | 19.25 |
| treatment 5 | 2053.4 | 1976.15 | 1929.68 | 1986.41 | 266.88 | 15.52 |

The three treatments in which Pseudomonas protegens was added had higher yield of garlic. Compared with treatment 1 as control (the farmers' customary fertilization), the percent increase of yield reached 9.21%, 19.25%, and 15.52%, respectively. The highest yield of garlic was resulted from the treatment with Pseudomonas protegens CHA0-ΔretS-NiF, which was 2050.54 kg/mu. The second highest yield of garlic was resulted from the treatment with Pseudomonas protegens while reducing nitrogen fertilizer. In said group, when the treatment with Pseudomonas protegens CHA0-ΔretS-NiF was applied while the nitrogen fertilizer was reduced by 30%, the yield of 1986.41 kg/mu was still obtained. Treatment with another marketed Pseudomonas protegens-Yun resulted the yield of 1877.16 kg/mu, and the percent increase of yield of 9.21%. The promotion effect of Pseudomonas protegens-Yun on the yield of garlic was not as good as that of Pseudomonas protegens CHA0-ΔretS-NiF engineered bacterium.

TABLE 5
Differences in yield of garlic sprouts of different treatments

| | | | | | | Percent |
|---|---|---|---|---|---|---|
| | Test | Test | Test | | Increase | increase |
| | yield 1 | yield 2 | yield 3 | Yield/mu | of yield | of yield |
| treatment | (kg/mu) | (kg/mu) | (kg/mu) | (kg/mu) | (kg/mu) | (%) |
| treatment 1 | 316.85 | 318.14 | 321.48 | 318.83 | | |
| treatment 2 | 321.97 | 352.39 | 316.18 | 330.18 | 11.35 | 3.56 |
| treatment 3 | 348.16 | 346.86 | 348.16 | 347.73 | 28.90 | 9.06 |
| treatment 4 | 364.83 | 371.50 | 390.89 | 375.74 | 56.92 | 17.85 |
| treatment 5 | 356.17 | 380.71 | 360.79 | 365.89 | 47.06 | 14.76 |

Among the five treatments, the three treatments with the addition of Pseudomonas protegens microbial agent had higher yields of garlic sprouts of 347.73 kg/mu, 375.74 kg/mu, and 365.89 kg/mu, which were higher than treatment 1 of the customary manner of farmers (increased by 9.06%, 17.85% and 14.76%, respectively). Judging from the yield of garlic sprouts, treatment with Pseudomonas protegens microbial agent could significantly promote the yield of garlic sprouts. Among them, the treatment which had the largest promotion effect was that with Pseudomonas protegens CHA0-ΔretS-NiF. The yield of garlic sprouts increased by 17.85% compared with the control. The yield of garlic sprouts of treatment 5 (Pseudomonas protegens CHA0-ΔretS-NiF while reducing nitrogen) was slightly less than treatment 4, but overall remained at a relatively average level.

It can be seen that the application of CHA0 microbial agents had a great impact on the quality of garlic. It can bring huge economic benefits to customers, and reduce ⅓ of the usage of nitrogen fertilizer. Under the premise of not affecting the quality of the product, the production cost of the customer can be reduced, and the effect was immediate.

Analysis of Garlic Disease Index in Different Experimental Treatments

The main diseases of garlic are leaf blight and root rot. Garlic leaf blight is one of the common diseases on garlic, and it occurs in different degrees in various vegetable areas, mainly harms garlic cultivated in open field. During the period when garlic grows, in the year having more and heavy rainfall, the disease is severe. Severe disease often causes the die of diseased leaves, premature senescence of plants, reduced garlic production, and rot of garlic, which directly affect yield.

The leaf blight disease in the middle stage can only be observed, and the disease index was calculated by counting the number of garlic diseases in the plot at the final harvest.

TABLE 6
Effects of different treatments on garlic diseases

| | | Number of | |
|---|---|---|---|
| | Number of | diseased | Disease |
| | garlic | garlic | index |
| Treatment | (number/2 m²) | (number/2 m²) | (%) |
| Treatment 1 | 87 | 8 | 9.19 |
| Treatment 2 | 82 | 7 | 8.53 |
| Treatment 3 | 95 | 6 | 6.32 |
| Treatment 4 | 98 | 4 | 4.08 |
| Treatment 5 | 94 | 5 | 5.32 |

Before sowing, treatments 4 and 5 to which Pseudomonas protegens was applied were not dressed with donine. In the later period, farmers applied pesticides four times totally but treatments 4 and 5 were not sprayed. From the final garlic harvest data, the disease index of treatment 4 was the lowest, and that of treatment 5 was also slightly lower than those of the farmers' convention as control and the optimized fertilization. This showed that Pseudomonas protegens had significant control effect on root rot of garlic.

The above description of the specific embodiments of the present invention has been described with reference to the accompanying drawings, but is not intended to limit the scope of the present invention. On the basis of the technical solutions of the present invention, various modifications or variations that can be made by the skilled in the art without any creative work are still within the scope of protection of the present invention.

1. A Pseudomonas protegens CHA0 mutant strain CHA0-ΔretS-NiF, which is deposited under the accession number CGMCC No. 14476.

2. A composition, such as a microbial agent, wherein its active ingredient is the Pseudomonas protegens mutant strain CHA0-ΔretS-NiF of claim 1.

3. Use of the Pseudomonas protegens mutant strain CHA0-ΔretS-NiF of claim 1 in killing bacteria in plants, fixing nitrogen, promoting plant growth, increasing plant yield, and/or controlling plant diseases.

4. A method for producing the Pseudomonas protegens mutant strain CHA0-ΔretS-NiF, which includes the following steps:
a) Knocking out retS gene in the genome of Pseudomonas protegens CHA0; and
b) Cloning the entire NiF nitrogen-fixing gene island in the genome of Pseudomonas stutzeri DSM4166 into the strain obtained in step a), and then heterologously expressing the same.

5. A method for killing bacteria in plants, fixing nitrogen, promoting plant growth, increasing plant yield, and/or controlling plant diseases, comprising administering to a plant or a seed thereof the Pseudomonas protegens mutant strain CHA0-ΔretS-NiF of claim 1.

6. A fermentation culture method of the Pseudomonas protegens mutant strain CHA0-ΔretS-NiF of claim 1, comprising the following steps:
(1) Seed activation: removing a glycerol tube containing the CHA0-ΔretS-NiF strain from a −80° C. ultra-low temperature freezer, after thawing, taking a small amount of bacterial solution and streaking it on a LB+genta20 plate, invertedly incubating it in an constant temperature biochemical incubator at 30° C. for 20 hours, and randomly selecting 5 single colonies from the plate to perform colony PCR verification to ensure that the correct target strain is obtained;
(2) Shake flask seed culture: inoculating the activated CHA0-ΔretS-NiF strain into KB medium and placing it in a full-temperature shaking incubator for 20 hours to obtain the seed solution;
(3) Fermenter culture: inoculating the seed solution into a fermenter containing KB medium at the inoculation amount of 5-10%, i.e., 5-10 ml seed solution per 100 ml of KB medium; after the inoculation, setting aeration volume, dissolved oxygen, temperature, rotation speed and pH, taking the bacterial solution every 6 h to measure the cell density, wherein the fermentation period is 96 h.

7. The fermentation culture method of claim 6, wherein the formula of the KB medium in steps (2) and (3) is: 10 mL glycerol, 20 g peptone, 1.5 g K₂HPO₄, 1.5 g MgSO₄.7H₂O per 1000 mL of water.

8. The fermentation culture method of claim 6, wherein the conditions of the culture in step (2) are 30° C. and 200 rpm.

9. The fermentation culture method of claim 6, wherein the conditions of the fermenter culture in step (3) are: a temperature of 26 to 32° C., a pH of 6 to 7.5, a rotation speed of 300 to 600 rpm, a ventilation volume of 0.8 to 4.0 L/min, and dissolved oxygen of 0.8-1.0 L/min; and the dissolved oxygen is not connected in series to the rotation speed; after culturing for 12-24 hours, 25-100 mL of 50 wt % glucose aqueous solution is added by flow; after that, 25-100 mL of 50% glucose aqueous solution is added by flow every 2 to 6 hours until the end of fermentation; during the period, 20 v/v % of phosphoric acid/ammonia is used to maintain a stable pH, and 50 v/v % of antifoaming agent is used for defoaming.

10. The fermentation culture method of claim 9, wherein the conditions of the fermenter culture in step (3) are: the temperature is 28° C., the pH is 7, and the rotation speed is 600 rpm.

11. A method for killing bacteria in plants, fixing nitrogen, promoting plant growth, increasing plant yield, and/or controlling plant diseases, comprising administering to a plant or a seed thereof the Pseudomonas protegens mutant strain CHA0-ΔretS-NiF of the composition of claim 2.


SEQUENCE LISTING (16 sequences)

SEQ ID NO: 1 - DNA, 83 bp, Artificial
  Primer 1
agtgaattgtaatacgactcactatagggcgaattcgagctcggtacccgcttaagtacggctacctggagctcgcgccagtg

SEQ ID NO: 2 - DNA, 114 bp, artificial
  Primer 2
tacggctacctggagctcgcgccagtgcttgccgacatcgaatcacggccgctgctgcagcacgtggtggtcaccggccgggatccgtttaaacacaaatggcaagggctaatg

SEQ ID NO: 3 - DNA, 114 bp, artificial
  Primer 3
attgatgttttccttggccagcgcctcgaacatccggctggcgacgcctgcgtgcgaacgcataccgacaccgacgatagggatccgtttaaacggtgtggtagctcgcgtatt

SEQ ID NO: 4 - DNA, 85 bp, artificial
  Primer 4
gcgacactatagaatactcaagcttggcatgaatgcaggtcgactctagagaatattgatgttttccttggccagcgcctcgaac

SEQ ID NO: 5 - DNA, 18 bp, artificial
  NiF-check-1
ggtctaccagctcgacct

SEQ ID NO: 6 - DNA, 20 bp, artificial
  NiF-check-2
cgattccagcgtcgaatgat

SEQ ID NO: 7 - DNA, 20 bp, artificial
  NiF-check-3
gctgacctccttgaggtgct

SEQ ID NO: 8 - DNA, 19 bp, artificial
  NiF-check-4
cagcggcacctcgaggagt

SEQ ID NO: 9 - DNA, 19 bp, artificial
  NiF-check-5
gatagagcaggtcctcgat

SEQ ID NO: 10 - DNA, 20 bp, artificial
  NiF-check-6
ggtgctctacgtcagccatt

SEQ ID NO: 11 - DNA, 20 bp, artificial
  NiF-check-7
cgacagatcctgattaccgt

SEQ ID NO: 12 - DNA, 21 bp, artificial
  NiF-check-8
taccctcgaccagcttgagca

SEQ ID NO: 13 - DNA, 21 bp, artificial
  check-5′
tgcttctaccgcaaggacatc

SEQ ID NO: 14 - DNA, 21 bp, artificial
  check-3′
gctgatgaagcacgagagcac

SEQ ID NO: 15 - DNA, 97 bp, artificial
  rets-genta-loxm-5′
gcacacgcccttgccgtgcggtcattacgccgcgcatagttataatcaggcatcaaccaacgaagggatttcgccagctgaattacattcccaaccg

SEQ ID NO: 16 - DNA, 98 bp, artificial
  rets-genta-loxm-3′
tggagcatggtgggagctcacgactaaaggagggcgagcgagagtttaacaggcgccgcagagcctgtcggctcacaacttaaatgtgaaagtgggtc